(19) United States
(12) Patent Application Publication
(10) Pub. No.: US 2009/0292194 A1
(43) Pub. Date: Nov. 26, 2009

(54) Chiropractic Care Management Systems and Methods

(75) Inventors: Imad Libbus, Saint Paul, MN (US); Yatheendhar D. Manicka, Woodbury, MN (US); Mark J. Bly, Falcon Heights, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/468,493

(22) Filed: May 19, 2009

Related U.S. Application Data
(60) Provisional application No. 61/055,638, filed on May 23, 2008.

(51) Int. Cl. A61B 5/04; A61B 5/11

(52) U.S. Cl. 600/391; 600/519; 600/595

(57) ABSTRACT

An adherent device may be placed on a patient's chest for monitoring heart rate variability for chiropractic care. The device may comprise an adherent patch configured to adhere to the patient continuously for an extended period, for example an extended period of one week, and the HRV can be determined for the extended period. Two or more electrodes may be used to measure a cardiac signal and determine the HRV. The device may comprise accelerometers to measure at least one of posture, flexion/extension or lateral movement of the patient. The device may be placed on the patient and used in the clinic, and the patient may be sent home from the clinic with the adherent device. The device may wirelessly transmit heart rate data to an external device, such as a handheld monitor, that the chiropractor may consult during treatment.

### CROSS-REFERENCES TO RELATED APPLICATIONS

The present application claims the benefit under 35 USC 119(e) of U.S. Provisional Application No. 61/055,638 filed May 23, 2008; the full disclosures of which are incorporated herein by reference in their entirety.

### BACKGROUND OF THE INVENTION

1. Field of the Invention

The present invention relates to patient monitoring and/or treatment. Although embodiments make specific reference to monitoring electrocardiogram signals with an adherent patch for chiropractic care, the systems, methods and devices described herein may be applicable to many applications in which physiological monitoring is used, for example wireless physiological monitoring for extended periods.

Patients are often treated for diseases and/or conditions associated with a compromised status of the patient, for example a compromised physiologic status. In some instances, a patient may report symptoms that require diagnosis to determine the underlying cause. For example, a patient may report fainting or dizziness that requires diagnosis, in which long term monitoring of the patient can provide useful information as to the physiologic status of the patient. In some instances a patient may have suffered a trauma such as a back injury that may require care and/or monitoring. One example of a device to provide long term monitoring of a patient is the Holter monitor, or ambulatory electrocardiography device.

In addition to measuring heart signals with electrocardiograms, known physiologic measurements include impedance measurements. For example, transthoracic impedance measurements can be used to measure hydration and respiration. Although transthoracic measurements can be useful, such measurements may use electrodes that are positioned across the midline of the patient, and may be somewhat uncomfortable and/or cumbersome for the patient to wear.

The chiropractic health professional may be concerned with the diagnosis, treatment and prevention of mechanical disorders of the musculoskeletal system. These disorders may have an effect on the function of the nervous system and on general health. In at least some instances, chiropractic treatment can be manual and may include spinal manipulation and/or adjustment. By restoring function to the musculoskeletal system, chiropractor care can play a role in relieving disorders and accompanying pain or discomfort, arising from accidents, stress, lack of exercise, poor posture, illness and everyday wear and tear. In some instances, the chiropractic professional may use heart rate variability (hereinafter “HRV”) to assess the condition of a patient.

Work in relation to embodiments of the present invention suggests that known methods and apparatus for monitoring and/or treating patients with chiropractic care may be less than ideal. Many devices that measure heart rate variability are connected to the patient, and in at least some instances, mobility of the patient may be limited while measurements are taken. At least some of the known wearable monitoring devices may not be suited for chiropractic care and may be somewhat uncomfortable, which may lead to patients not wearing the devices and not complying with direction, such that data collected may be less than ideal. Although implantable devices are known, many of these devices can be invasive and/or costly, and may suffer at least some of the shortcomings of known wearable devices.

Therefore, a need exists for improved patient monitoring and treatment with chiropractic care. Ideally, such improved patient monitoring and treatment would avoid at least some of the short-comings of the present methods and devices.

2. Description of the Background Art

The following U.S. patents and applications make reference to heart rate variability, heart rate variation or heart rate variance: 2007/0167848; 2006/0281996; 2006/0009701; 2005/0148895; 2005/0131288; 2005/0124901; U.S. Pat. Nos. 7,160,253; and 6,775,566. U.S. Pat. No. 7,156,808 makes reference to wireless patient monitoring.

### BRIEF SUMMARY OF THE INVENTION

The present invention relates to patient monitoring and/or treatment. Although embodiments make specific reference to monitoring electrocardiogram signals with an adherent patch for chiropractic care, the systems, methods and devices described herein may be applicable to many applications in which physiological monitoring is used, for example wireless physiological monitoring for extended periods.

Embodiments of the present invention are generally directed to an adherent or wearable device for monitoring a patient's heart rate variability for chiropractic care. The device may be used in a clinic or home setting to determine the effectiveness of chiropractic therapy. Chiropractors may use heart rate variability as an index of a patient's well-being, and therapy can be adjusted and optimized to enhance a patient's heart rate variability. The range of motion of the patient may be measured, for example head motion, with at least one accelerometer. The adherent or wearable device may be placed on a patient's chest for monitoring heart rate variability. In some embodiments, the device may comprise an adherent patch configured to adhere to the patient continuously for an extended period, for example an extended period of one week. The heart rate variability can be determined for the extended period. Such extended monitoring can be beneficial as the heart rate variability and/or patient motion can be monitored for actual patient activities, such as exercise, work, sitting and sleep. Thus, the heart rate variability and/or patient motion that occurs with actual patient activities can be used to diagnose and/or treat the patient, which may provide an improved assessment of the patient. Two or more electrodes may be used to measure a cardiac signal and determine the heart rate variability, and at least one accelerometer can be used to measure patient motion. The device may be placed on the patient and used in the clinic, and the patient may be sent home from the clinic with the adherent device. The device may wirelessly transmit heart rate data to an external device, such as a handheld monitor, that the chiropractor may consult during treatment. The device may collect data for several days and may transmit data through a wireless modem back to the clinic. In some embodiments, the data can be stored on the device for subsequent retrieval. The device may monitor respiration rate and/or respiration rate variability, and may also perform cardiac rhythm monitoring. The device may be used as part of a care management system that comprises an algorithm for chiropractic care based on heart rate variability response.

In a first aspect, embodiments of the present invention provide an adherent device for chiropractic monitoring of a patient. The device comprises an adhesive patch to adhere to a skin of the patient. At least two electrodes are connected to the patch and capable of electrically coupling to the patient. Electrocardiogram circuitry is coupled to the at least two electrodes to measure an electrocardiogram signal of the patient. A processor comprising a tangible medium is coupled to the electrocardiogram circuitry, the processor comprising a tangible medium configured to determine at least one of a heart rate or a heart rate variability of the patient in response to the electrocardiogram signal.

In many embodiments, the adhesive patch is configured to mechanically couple the at least two electrodes to the skin and obtain the electrocardiogram signal for at least one week.

In many embodiments, the processor is configured to determine the heart rate variability with at least one of a time domain determination, a frequency domain determination or a non-linear determination.

With respect to frequency domain determination, the processor can be configured to determine the heart rate variability in response to at least one of a low frequency from about 0.04 to 0.15 Hz or a high frequency from about 0.15 Hz to about 0.4 Hz. The processor may be configured to determine the heart rate variability with the frequency domain determination in response to a ratio of a low frequency band comprising at least one low frequency from about 0.04 to 0.15 Hz and a high frequency band comprising at least one high frequency from about 0.15 Hz to about 0.4 Hz.

With respect to time domain determination, the processor can be configured to determine the heart rate variability with the time domain determination in response to a standard deviation of R-R intervals. The processor can be configured to determine R-R intervals based on from about one to ten minutes of the electrocardiogram signal. The heart rate variability may comprise a standard deviation of the R-R intervals, and the processor can be configured to determine the heart rate variability several times over the at least one week. The processor may be configured to determine averages of R-R intervals from the electrocardiogram signal, and the processor can be configured to determine each of the averages of the R-R intervals based on from about one to ten minutes of the electrocardiogram signal. The heart rate variability may comprises a standard deviation of the averages of the R-R intervals, and the processor may be configured to determine the heart rate variability several times over the at least one week.

In many embodiments, the processor can be configured to determine the heart rate variability at least once per hour for each hour of the at least one week.

In many embodiments, the adhesive patch is mechanically coupled to the at least two electrodes and the electrocardiogram circuitry to support the at least two electrodes and the electrocardiogram circuitry when the adherent patch is adhered to the skin of the patient.

In many embodiments, the device comprises wireless communication circuitry to transmit the heart rate variability to a caregiver computer system with a communication protocol. The communications protocol may comprise a two way protocol such that the caregiver computer system is capable of issuing commands to the processor to control data collection. The processor can be configured to transmit the at least one of the heart rate or the heart rate variability to the caregiver computer system in response to a command from the caregiver computer system when the wireless communication circuitry is located in an office of the caregiver. The caregiver computer system may comprise a display visible to a caregiver and a tangible medium configured to show information on the display in response to the electrocardiogram signal.

In many embodiments, the wireless communication circuitry is configured to communicate with a remote center using an intermediate device.

The adherent device may comprise wireless communication circuitry to transmit the at least one of the heart rate or the heart rate variability to a remote center with a communication protocol. The wireless communication circuitry may be configured to transmit the electrocardiogram signal to the remote center with an intermediate device. The communication protocol may comprise at least one of Bluetooth, Zigbee, WiFi, WiMax, IR, a cellular protocol, amplitude modulation or frequency modulation. The intermediate device may comprise a data collection system to collect and/or store data from the wireless transmitter and wherein the data collection system is configured to communicate periodically with the remote center with wireless connection and/or wired communication. The communications protocol may comprise a two way protocol such that the remote center is capable of issuing commands to the processor to control data collection.

In many embodiments, the processor is configured to control collection and transmission of data from the electrocardiogram circuitry.

In many embodiments, the adherent patch comprises a breathable tape, the breathable tape comprising a breathable material with an adhesive.

In many embodiments, the adherent device may comprise an accelerometer connected to the adhesive patch to measure at least one of a rotation, a flexion/extension, a lateral movement or a posture of the patient. The accelerometer may be connected to a second adhesive patch configured for placement on at least one of a head, a neck or an ear of the patient.

In another aspect, embodiments of the present invention provide an adherent device system for chiropractic monitoring of a patient. The system comprises at least one adhesive patch to adhere to a skin of the patient, and at least accelerometer connected to the at least one patch to generate at least one accelerometer signal. A processor is coupled to the at least one accelerometer, and the processor comprises a tangible medium configured to determine at least one of a rotation, a flexion/extension, a lateral movement or a posture of the patient in response to the at least one accelerometer signal.

In another aspect, embodiments of the present invention provide a method of monitoring a patient. The method comprises adhering an adhesive patch to a skin of the patient to couple at least two electrodes to the skin of the patient. An electrocardiogram signal of the patient is measured with electrocardiogram circuitry coupled to at least two of the at least two electrodes. At least one of a heart rate or a heart rate variability of the patient is determined.

In many embodiments, the patch is adhered to the patient for at least one week and the heart rate or the heart rate variability is determined for the at least one week.

In many embodiments, averages of R-R intervals are determined from the electrocardiogram signal, and each of the averages of the R-R intervals are determined based on from about one to ten minutes of the electrocardiogram signal. The heart rate variability may comprise a standard deviation of the averages of the R-R intervals, and the heart rate variability may be determined several times over the at least one week. For example, the heart rate variability may be determined at least once per hour for each hour of the at least one week.

In many embodiments, the adhesive patch may support the at least two electrodes and the processor when the adherent patch is adhered to the skin of the patient.

In another aspect, embodiments of the present invention provide a method of chiropractic monitoring a patient. At least one adhesive patch is adhered to a skin of the patient to couple at least one accelerometer to the skin of the patient. At least one accelerometer signal of the patient is measured with the at least one accelerometer coupled to the skin of the patient. At least one of a rotation, a flexion/extension, a lateral movement or a posture of the patient is determined in response to the accelerometer signal.

In another aspect, embodiments of the present invention provide an adherent device to monitor a patient for an extended period. The device comprises a breathable tape, and the breathable tape comprises a porous material with an adhesive coating to adhere the breathable tape to a skin of the patient. At least one electrode is affixed to the breathable tape and capable of electrically coupling to a skin of the patient. At least one gel may be disposed over a contact surface of the at least one electrode to electrically connect the electrode to the skin. A circuit board can be connected to the electrodes to couple the printed circuit board to the electrodes. Electronic components can be electrically connected to the printed circuit board and coupled to the at least one electrode to measure an electrocardiogram signal of the patient. A processor may be coupled to the electronic components to determine at least one of a heart rate or a heart rate variability of the patient.

In many embodiments, a breathable cover is disposed over the circuit board and electronic components and connected to at least one of the electronics components, the printed circuit board or the breathable tape. An electronics housing can be adhered to at least one of the electronics components or the printed circuit board, such that the electronics housing is disposed between the cover and electronics components.

In many embodiments, a gel cover is positioned over the breathable tape to control gel hydration and to inhibit a flow of the gel through the breathable tape. The printed circuit board may be located over the gel cover such that the gel cover is disposed between the breathable tape and the printed circuit board. The breathable tape comprises a first porosity and the gel cover comprises a breathable tape with a second porosity, the second porosity less than the first porosity to decrease a flow of moisture to and from the at least one gel and to decrease flow of the gel through the breathable tape. The breathable tape, the adhesive coating and the at least one electrode may be separable from the printed circuit board and electronic components such that the printed circuit board, electronic components, housing and cover are reusable.

In many embodiments, the at least one electrode extends through at least one aperture in the breathable tape.

### BRIEF DESCRIPTION OF THE DRAWINGS

FIG. 1A shows a patient and a monitoring system comprising an adherent device, according to embodiments of the present invention;

FIG. 1A1 shows an adherent device system comprising a plurality of adherent devices simultaneously adhered to the patient, according to embodiments of the present invention;

FIG. 1A1-1 shows detail of second adherent device as in FIG. 1A1;

FIG. 1B shows a bottom view of the adherent device as in FIG. 1A comprising an adherent patch;

FIG. 1B1 shows a bottom view of an adherent patch similar to the patch of FIG. 1B and comprising at least four electrodes for measuring impedance, according to embodiments of the present invention;

FIG. 1C shows a top view of the adherent patch, as in FIG. 1B;

FIG. 1D shows a printed circuit boards and electronic components over the adherent patch, as in FIG. 1C;

FIG. 1D1 shows an electrocardiogram signal measured with ECG circuitry, according to embodiments of the present invention;

FIG. 1E shows batteries positioned over the printed circuit board and electronic components as in FIG. 1D;

FIG. 1F shows a top view of an electronics housing and a breathable cover over the batteries, electronic components and printed circuit board as in FIG. 1E;

FIG. 1G shows a side view of the adherent device as in FIGS. 1A to 1F;

FIG. 1H shown a bottom isometric view of the adherent device as in FIGS. 1A to 1G;

FIGS. 1I and 1J show a side cross-sectional view and an exploded view, respectively, of the adherent device as in FIGS. 1A to 1H;

FIG. 1K shows at least one electrode configured to electrically couple to a skin of the patient through a breathable tape, according to embodiments of the present invention; and

FIG. 2A shows a method of determining heart rate variability of a patient, according to embodiments of the present invention.

### DETAILED DESCRIPTION OF THE INVENTION

The present invention relates to patient monitoring and/or treatment. Although embodiments make specific reference to monitoring electrocardiogram signals with an adherent patch for chiropractic care, the systems, methods and devices described herein may be applicable to many applications in which physiological monitoring is used, for example wireless physiological monitoring for extended periods.

Embodiments of the present invention is generally directed to an adherent or wearable device for monitoring a patient's heart rate variability (HRV) for chiropractic care. The device may be used in a clinic or home setting to determine the effectiveness of chiropractic therapy. Chiropractors use heart rate variability as an index of a patient's well-being, and therapy can be adjusted and optimized to enhance a patient's HRV. The adherent or wearable device can be placed on at least one of the patient's chest, ribs or back for monitoring heart rate variability. The adherent device may be configured to adhere to the skin of the patient with an adherent patch, for example breathable tape, coupled to at least two electrodes. The device may comprises electrocardiogram circuitry coupled to the at least two electrodes, and the circuitry can measure electrocardiogram signal to determine the heart rate variability (hereinafter “HRV”) of the patient. The heart rate variability can be determined in many ways, for example with at least one of frequency determination, non-linear determination or time domain determination. With time domain determination, the heart rate intervals may be determined, for example R-R intervals. The device may be placed on the patient and used in the clinic. The device can wirelessly transmit heart rate data to an external device, such as a handheld monitor, that the chiropractor may consult during treatment. Alternatively or in addition, the patient may be sent home with the device, which can collect data for several days, for example one week, and may transmit data through a wireless modem back to the clinic. In some embodiments, the data can be stored on the device for subsequent retrieval. The device may monitor respiration rate and/or respiration rate variability. The device may also perform cardiac rhythm monitoring, and may be used as part of a care management system that comprises an algorithm for chiropractic care based on HRV response.

The adherent devices described herein may be used for 90 day monitoring, or more, and may comprise completely disposable components and/or reusable components, and can provide reliable data acquisition and transfer. In many embodiments, the patch is configured for patient comfort, such that the adherent patch can be worn and/or tolerated by the patient for extended periods, for example 90 days or more. The patch may be worn continuously for at least seven days, for example 14 days, and then replaced with another patch. Adherent devices with comfortable patches that can be worn for extended periods and in which patches can be replaced and the electronics modules reused. In many embodiments, the adherent patch comprises a tape, which comprises a material, preferably breathable, with an adhesive, such that trauma to the patient skin can be minimized while the patch is worn for the extended period. The printed circuit board may comprise a flex printed circuit board that can flex with the patient to provide improved patient comfort.

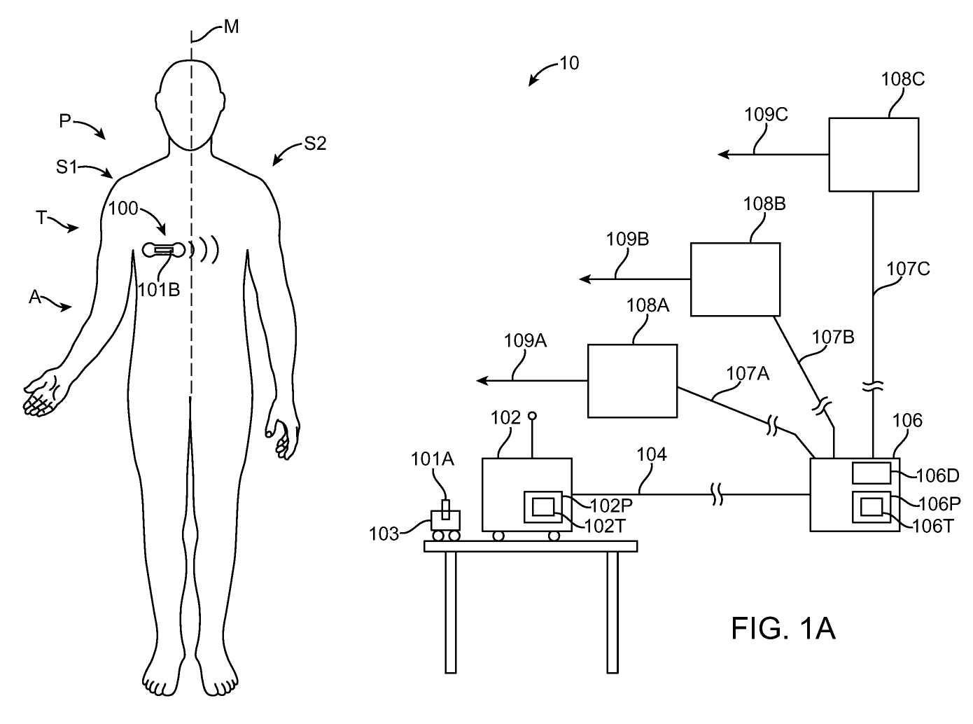

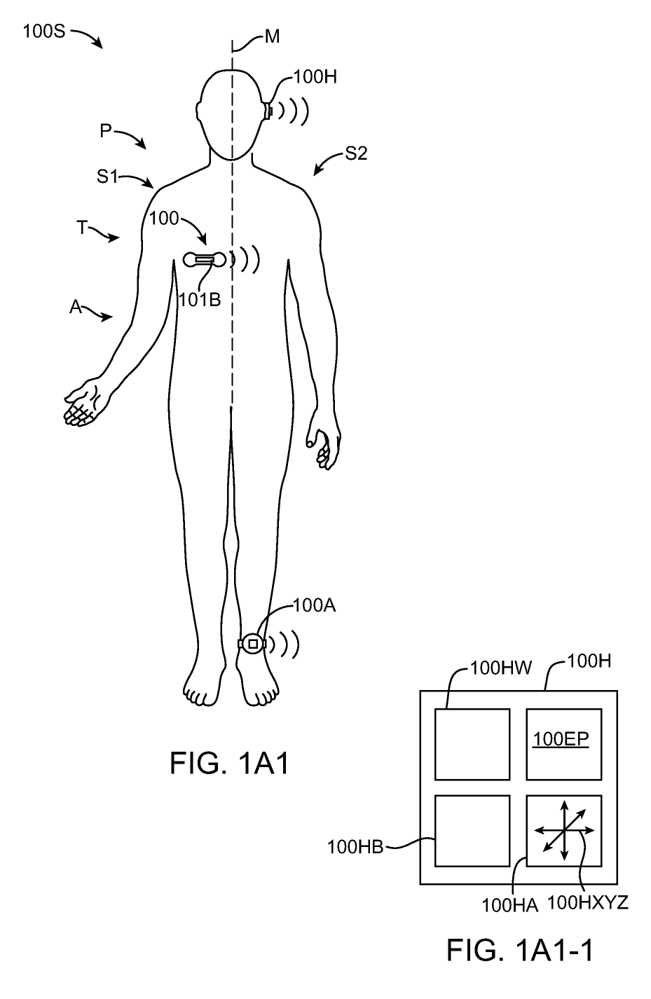

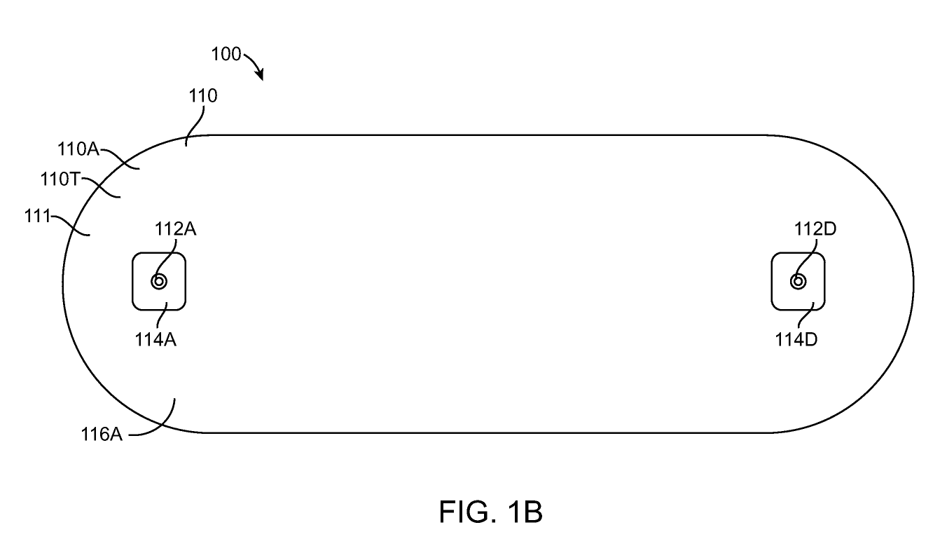

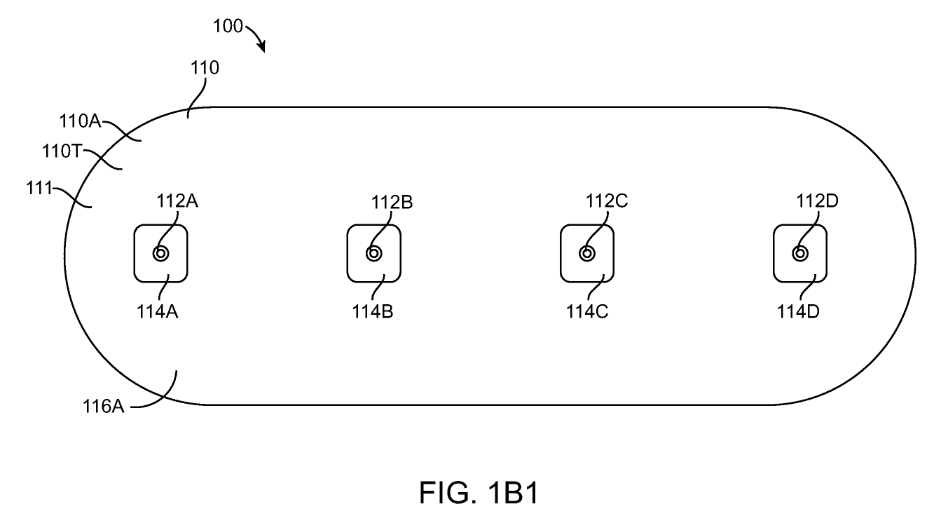

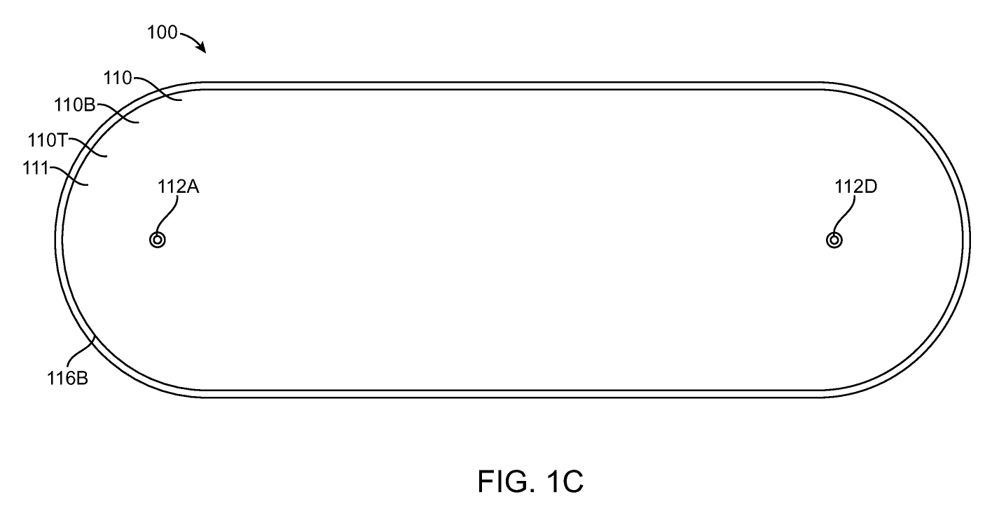

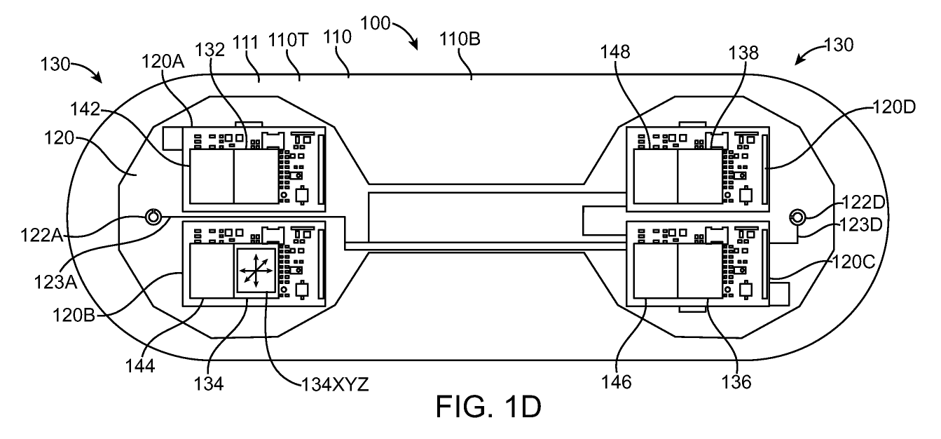

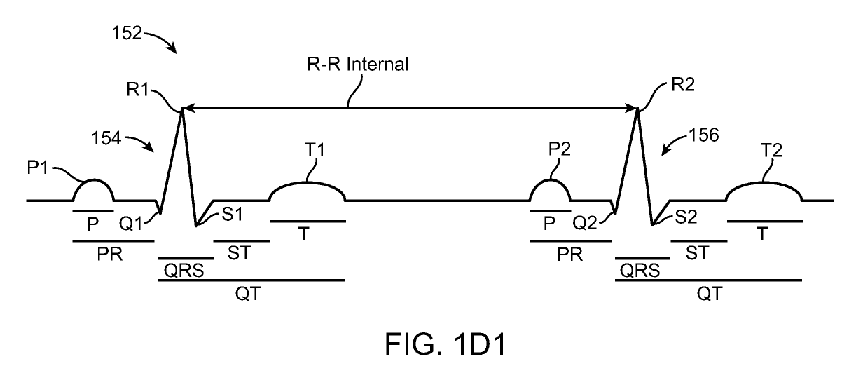

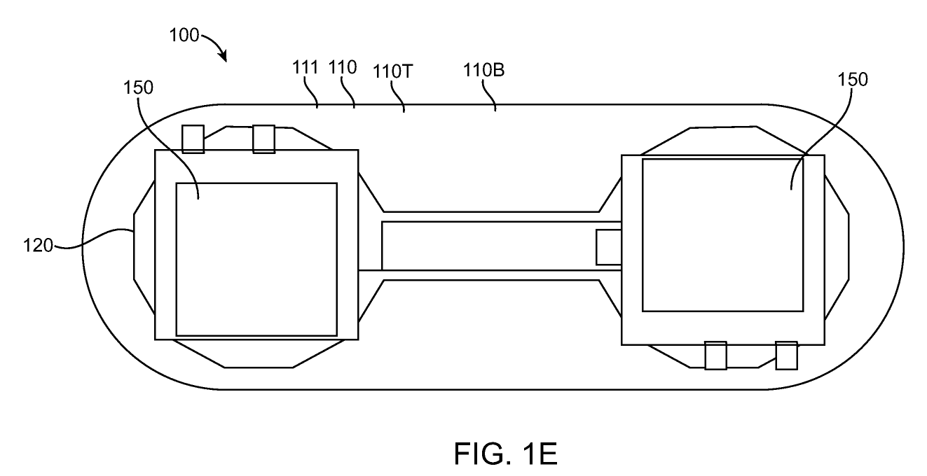

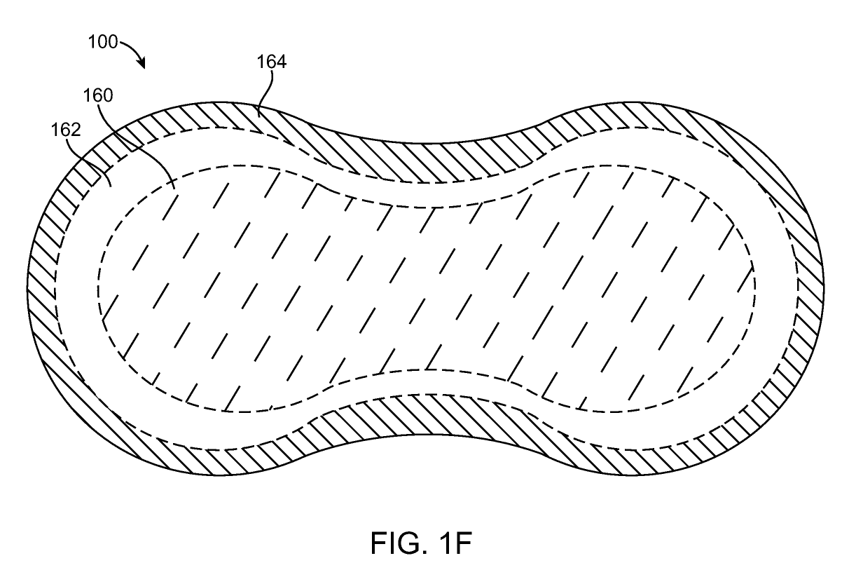

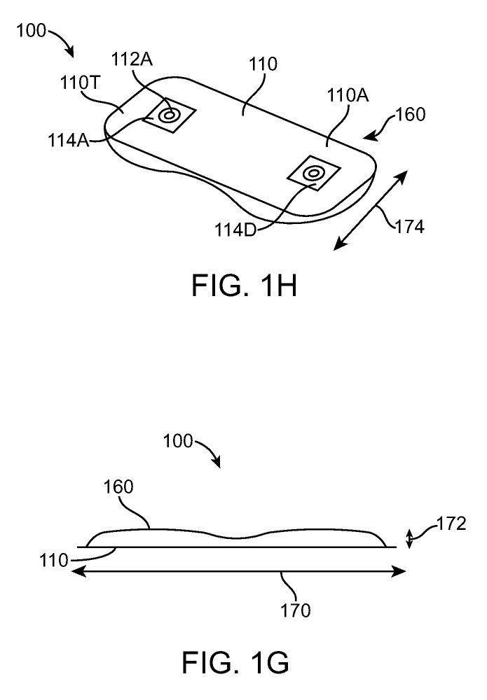

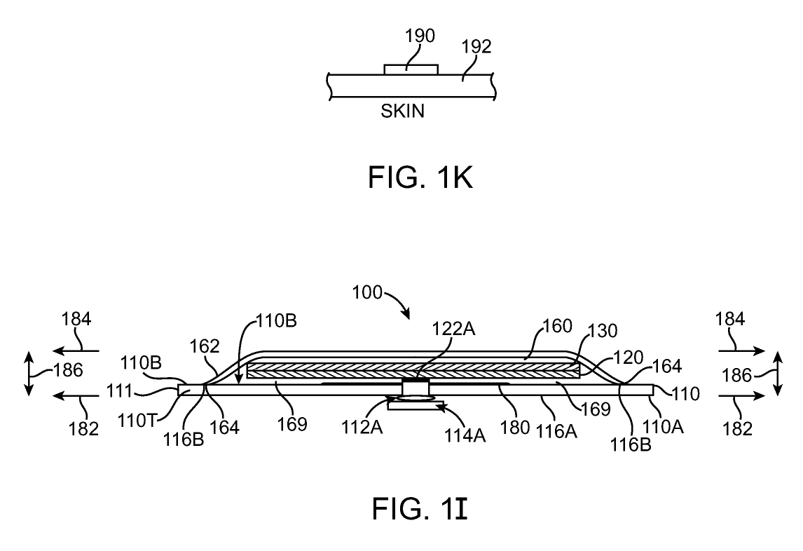

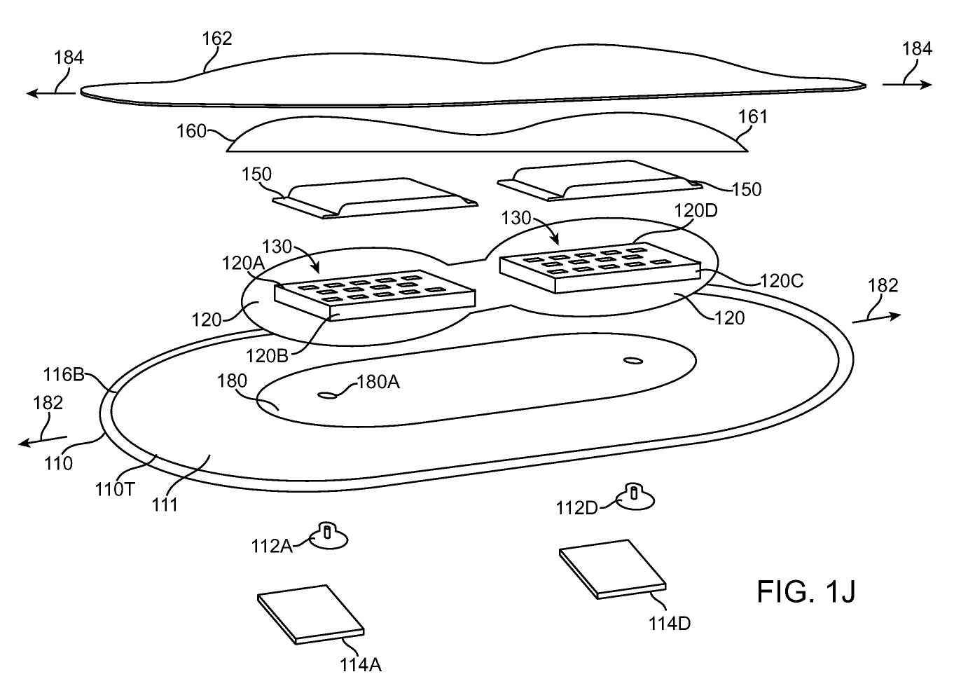

FIG. 1A shows a patient P and a monitoring system 10. Patient P comprises a midline M, a first side S1, for example a right side, and a second side S2, for example a left side. Monitoring system 10 comprises an adherent device 100. Adherent device 100 can be adhered to a patient P at many locations, for example thorax T of patient P. In many embodiments, the adherent device may adhere to one side of the patient, from which side data can be collected. Work in relation with embodiments of the present invention suggests that location on a side of the patient can provide comfort for the patient while the device is adhered to the patient.

Monitoring system 10 includes components to transmit data to a computer system 106. Computer system 106 can be located in the same building as the patient. For example, computer system 106 can be located in an office of the health care provider, such as the office of the chiropractor. In some embodiments, computer system 106 can be located as far from the patient as a separate continent from the patient, for example the patient located on a first continent and the computer system located on a second continent.

Adherent device 100 can communicate wirelessly to an intermediate device 102, for example with a single wireless hop from the adherent device on the patient to the intermediate device. Intermediate device 102 can communicate with computer system 106 in many ways, for example with a wireless connection 104, an internet connection and/or with a cellular connection. Intermediate device 102 can be located in the chiropractor's office to receive patient data stored on the adherent device, for example data stored over a one week period between visits. Intermediate device 102 can be located in the home of the patient and send data to the chiropractor's office. In some embodiments, intermediate device 102 comprises a plurality of intermediate devices with a first intermediate device disposed at the chiropractor's office and a second intermediate device disposed at the patient's home. In many embodiments, monitoring system 10 comprises a distributed processing system with at least one processor comprising a tangible medium of device 100, at least one processor 102P of intermediate device 102, and at least one processor 106P of computer system 106, each of which processors can be in electronic communication with the other processors. At least one processor 102P comprises a tangible medium 102T, and at least one processor 106P comprises a tangible medium 106T. Remote processor 106P may comprise a backend server located at the computer system.

Computer system 106 may comprise a display 106D for the healthcare provider to view patient data, for example for the chiropractor to view heart rate variability measured from the patient. Display 106D can be located in the chiropractor's office to allow chiropractor to view patient data when treating the patient. In some embodiments, the patient information can be sent to the health care provider at a location remote from the patient, for example when the patient and health care provider are located in separate buildings. Patient data can be sent to a handheld device to allow remote treatment of the patient.

Computer system 106 can be in communication with a health care provider 108A with a communication system 107A, such as the Internet, an intranet, phone lines, wireless and/or satellite phone. Health care provider 108A, for example a chiropractor's assistant, can be in communication with patient P with a communication system, for example with a two way communication system, as indicated by arrow 109A, for example by cell phone, email, landline. Computer system 106 can be in communication with a health care professional, for example a chiropractor 108B, with a communication system 107B coupled with a handheld device, such as the Internet, an intranet, phone lines, wireless and/or satellite phone. Chiropractor 108B can be in communication with patient P with a communication system comprising a handheld device, for example with a two way communication system, as indicated by arrow 109B, for example by cell phone, email, landline. Thus, in many embodiments, monitoring system 10 comprises a closed loop system in which patient care can be monitored and implemented from the computer system in response to signals from the adherent device.

In many embodiments, computer system 106 receives the patient data and applies a patient evaluation algorithm, for example an algorithm to calculate the heart rate variability from an electrocardiogram signal of the adherent device. Computer system 106, and/or the processor of the adherent device, can determine the heart rate variability in many ways, for example with at least one of time domain determination, frequency domain determination or non-linear determination.

Time Domain

Time domain measure of the heart rate variability may comprise the calculation of the standard deviation of beat-to-beat intervals. In other words the time intervals between heart beats can be statistically analyzed to obtain information about the autonomic nervous system. Other time domain measures of heart rate variability may include root mean square of the differences between heart beats (rMSSD), NN50 or the number of normal to normal complexes that fall within 50 milliseconds, and pNN50 or the percentage of total number beats that fall with 50 milliseconds.

Frequency Domain

A frequency domain method may comprise the application of the discrete Fourier transform to the beat-to-beat interval time series. This provides an estimation of the amount of variation at specific frequencies. Several frequency bands of interest have can be used in humans.

High Frequency band (HF) between about 0.15 and 0.4 Hz. HF may be driven by respiration and may derive mainly from vagal activity or the parasympathetic nervous system.

Low Frequency band (LF) between 0.04 and 0.15 Hz. LF may derive from both parasympathetic and sympathetic activity and can reflect the delay in the baroreceptor loop.

Very Low Frequency band (VLF) band between 0.0033 and 0.04 Hz. The origin of VLF may be attributed to thermal regulation of the body's internal systems.

Ultra Low Frequency (ULF) band between 0 and 0.0033 Hz. The major background of ULF may comprise day/night variation and therefore may be expressed in 24-hour recordings.

The ratio of low-to-high frequency spectra power (LF/HF) can be used as an index of sympathetic to parasympathetic balance of heart rate fluctuation, but this remains controversial because of still little understanding of the LF component, which may be affected by centrally generated brainstem rhythms, baro-reflex influences, as well as both sympathetic and parasympathetic inputs, etc.

Non-Linear

The non-linear method of analyzing heart rate variability may comprise the Poincaré Plot. The Poincaré plot can fit heart rate data points to an ellipse that is fitted to two intersecting lines. SD1 and SD2, or the standard deviations of the data points have also been applied in the context of Poincaré analysis.

The adherent device may be affixed and/or adhered to the body in many ways. For example, with at least one of the following: an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide|remove old patch|place new patch|remove guide), or a keyed attachment for chatter reduction. The patch and/or device may comprise an adhesiveless embodiment (e.g. chest strap), and/or a low-irritation adhesive for sensitive skin. The adherent patch and/or device can comprise many shapes, for example at least one of a dogbone, an hourglass, an oblong, a circular or an oval shape.

In many embodiments, the adherent device may comprise a reusable electronics module with replaceable patches, and each of the replaceable patches may include a battery. The module may collect cumulative data for approximately 90 days and/or the entire adherent component (electronics+patch) may be disposable. In a completely disposable embodiment, a “baton” mechanism may be used for data transfer and retention, for example baton transfer may include baseline information. In some embodiments, the device may have a rechargeable module, and may use dual battery and/or electronics modules, wherein one module 101A can be recharged using a charging station 103 while the other module 101B is placed on the adherent patch with connectors. In some embodiments, the intermediate device 102 may comprise the charging module, data transfer, storage and/or transmission, such that one of the electronics modules can be placed in the intermediate device for charging and/or data transfer while the other electronics module is worn by the patient.

System 10 can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, hear rate variability (HRV), heart rate turbulence (HRT), heart sounds (e.g. S3), respiratory sounds, blood pressure, activity, posture, wake/sleep, orthopnea, temperature/heat flux, and weight. The activity sensor may comprise one or more of the following: ball switch, accelerometer, minute ventilation, HR, bioimpedance noise, skin temperature/heat flux, BP, muscle noise, posture.

The adherent device can wirelessly communicate with computer system 106. The communication may occur directly (via a cellular or Wi-Fi network), or indirectly through intermediate device 102. Intermediate device 102 may consist of multiple devices, which can communicate wired or wirelessly to relay data to computer system 106.

In many embodiments, instructions are transmitted from computer system 106 to a processor supported with the adherent patch on the patient, and the processor supported with the patient can receive updated instructions for the patient treatment and/or monitoring, for example while worn by the patient.

FIG. 1A1 shows an adherent device system 100S comprising a plurality of adherent devices simultaneously adhered to the patient, for example adherent device 100, second adherent device 100H and third adherent device 100A. Adherent device system 100S may comprise wireless communication between and/or among devices adhered to the patient. Adherent device system 100S may comprise a component of system 10 described above. Second adherent device 100H can be positioned on at least one of the head or neck of the patient, for example on or behind the ear, to detect head movement and/or orientation, for example rotation of the head. Second adherent device 100H may comprise an earpiece, for example an ear piece configured to fit in an ear canal of the patient or fit on or behind a pinna of the patient for minimal visibility. Second adherent device 100H may comprise an accelerometer such as a position sensitive 3D accelerometer to generate an accelerometer signal so as to detect patient head orientation and/or movement. Third adherent device 100A may be disposed on the patient to detect full body rotation of the patient from the head to the ankle. Third adherent device 100A may comprise an accelerometer position sensitive 3D accelerometer to generate an accelerometer signal so as to detect patient leg movement and/or orientation to determine orientation of the foot, leg and/or ankle relative to the head. Adherent device 100 may comprise an accelerometer to detect patient motion and/or orientation, for example motion and/or orientation of the thorax in relation to the head and/or ankle.

FIG. 1A1-1 shows detail of second adherent device 100H. Second adherent device 100H may comprise a wireless communication circuitry 100HW, at least one battery 100HB, a processor 100HP and an accelerometer 100HA. Accelerometer 100HA may comprise a 3D accelerometer 100HXYZ sensitive to gravity and configured to generate an accelerometer signal so as to measure at least one of head rotation, head position or head inclination. Processor 100EP can process signals and/or data from the accelerometer. Wireless communication circuitry 100HW can transmit the data to other components of system 10, for example device 100 and/or intermediate device 102. Second adherent device 100H can attach to the head of the patient in many ways, for example at least one of on the ear, in the ear, behind the ear or on the jaw. Third adherent device 100A may comprise similar components.

The accelerometers described herein can be used in many ways to evaluate the patient. For example, posture of the patient can be monitored. Patients with back problems can be monitored to see how long they can sit, and in what position they sit. Sitting posture that is irregular may indicate that the patient has limited motion and/or pain and may indicate that sitting causes stress to the back. Such irregularities can be detected by comparing orientation of the accelerometers of the system, for example of device 100 and second device 100H.

Patient movement and/or range of motion can also be evaluated with a plurality of accelerometers adhered and/or attached to the patient. For example side to side bending of the patient can be measured to determine a side to side range of motion of the patient. Patient flexion and extension, for example up and down, can be measured to determine the range of flexion and/or extension motion. Such measurements can be made at baseline and monitored over time to evaluate a change in patient condition.

FIG. 1B shows a bottom view of adherent device 100 as in FIG. 1A comprising an adherent patch 110. Adherent patch 110 comprises a first side, or a lower side 110A, that is oriented toward the skin of the patient when placed on the patient. In many embodiments, adherent patch 110 comprises a tape 110T which is a material, preferably breathable, with an adhesive 116A. Patient side 110A comprises adhesive 116A to adhere the patch 110 and adherent device 100 to patient P. Electrodes 112A and 112D are affixed to adherent patch 110. In many embodiments, at least two electrodes are attached to the patch. The patch may comprise two electrodes to measure the electrocardiogram (ECG) of the patient. Gel 114A and gel 114D can each be positioned over electrodes 112A and 112D, respectively, to provide electrical conductivity between the electrodes and the skin of the patient. In many embodiments, the electrodes can be affixed to the patch 110, for example with known methods and structures such as rivets, adhesive, stitches, etc. In many embodiments, patch 110 comprises a breathable material to permit air and/or vapor to flow to and from the surface of the skin.

FIG. 1B-1 shows a bottom view of adherent patch 110 with at least four electrodes for measuring impedance. In addition to electrodes 112A and 112D, as described above, the adherent patch may comprise electrodes 112B and 112C. Although four electrodes are shown, some embodiments may comprise, for example, three electrodes. Four electrodes, for example electrodes 112A, 112B, 112C and 112D, can be used to measure hydration of the patient, for example with impedance measurements. The gel 114B and gel 114C can be disposed over electrodes 112B and 112C, respectively.

FIG. 1C shows a top view of the adherent patch 100, as in FIG. 1B. Adherent patch 100 comprises a second side, or upper side 110B. In many embodiments, electrodes 112A and 112D extend from lower side 110A through adherent patch 110 to upper side 110B. An adhesive 116B can be applied to upper side 110B to adhere structures, for example a breathable cover, to the patch such that the patch can support the electronics and other structures when the patch is adhered to the patient. The PCB may comprise completely flex PCB, rigid PCB, rigid PCB combined flex PCB and/or rigid PCB boards connected by cable.

FIG. 1D shows a printed circuit boards and electronic components over adherent patch 110, as in FIGS. 1A to 1C. In some embodiments, a printed circuit board (PCB), for example flex printed circuit board 120, may be connected to electrodes 112A and 112D with connectors 122A and 122D. Flex printed circuit board 120 can include traces 123A and 123D that extend to connectors 122A and 122D, respectively, on the flex PCB. Connectors 122A and 122D can be positioned on flex printed circuit board 120 in alignment with electrodes 112A and 112D so as to electrically couple the flex PCB with the electrodes. In some embodiments, connectors 122A and 122D may comprise insulated wires and/or a film with conductive ink that provide strain relief between the PCB and the electrodes. For example, connectors 122A and 122D may comprise a flexible polyester film coated with conductive silver ink. In some embodiments, additional PCB's, for example rigid PCB's 120A, 120B, 120C and 120D, can be connected to flex printed circuit board 120. Electronic components 130 can be connected to flex printed circuit board 120 and/or mounted thereon. In some embodiments, electronic components 130 can be mounted on the additional PCB's.

Electronic components 130 comprise components to take physiologic measurements, transmit data to computer system 106 and receive commands from computer system 106. In many embodiments, electronics components 130 may comprise known low power circuitry, for example complementary metal oxide semiconductor (CMOS) circuitry components. Electronics components 130 may comprise an activity sensor and activity circuitry 134, impedance circuitry 136 and ECG circuitry 136. In some embodiments, electronics circuitry 130 may comprise a microphone and microphone circuitry 142 to detect an audio signal from within the patient, and the audio signal may comprise a heart sound and/or a respiratory sound, for example an S3 heart sound and a respiratory sound with rales and/or crackles.

Electronics circuitry 130 may comprise a temperature sensor, for example a thermistor in contact with the skin of the patient, and temperature sensor circuitry 144 to measure a temperature of the patient, for example a temperature of the skin of the patient. A temperature sensor may be used to determine the sleep and wake state of the patient. The temperature of the patient can decrease as the patient goes to sleep and increase when the patient wakes up.

Electronics circuitry 130 may comprise a processor 146. Processor 146 comprises a tangible medium, for example read only memory (ROM), electrically erasable programmable read only memory (EEPROM) and/or random access memory (RAM). Processor 146 may comprise many known processors with real time clock and frequency generator circuitry, for example the PIC series of processors available from Microchip, of Chandler Ariz. In some embodiments, processor 136 may comprise the frequency generator and real time clock. The processor can be configured to control a collection and transmission of data from the impedance circuitry electrocardiogram circuitry and the accelerometer. In many embodiments, device 100 comprise a distributed processor system, for example with multiple processors on device 100.

Electronics circuitry 130 may comprise electromyogram (hereinafter “EMG”) circuitry 148 to measure muscle activity. EMG circuitry 148 can measure signals from muscles and may be connected to and/or comprise at least two of electrode 112A, electrode 112B, electrode 112C or electrode 112D. EMG circuitry 148 comprises an amplifier to amplify signals from contracting muscles so as to generate an EMG signal. EMG circuitry 148 can be connected to processor to send the EMG signal to the processor for storage and/or analysis.

In many embodiments, electronics components 130 comprise wireless communications circuitry 132 to communicate with computer system 106. The wireless communication circuitry can be coupled to the impedance circuitry, the electrocardiogram circuitry and the accelerometer to transmit to a computer system with a communication protocol at least one of the hydration signal, the electrocardiogram signal or the inclination signal. In specific embodiments, wireless communication circuitry is configured to transmit the hydration signal, the electrocardiogram signal and the inclination signal to the computer system with a single wireless hop, for example from wireless communication circuitry 132 to intermediate device 102. The communication protocol comprises at least one of Bluetooth, Zigbee, WiFi, WiMax, IR, amplitude modulation or frequency modulation. In many embodiments, the communications protocol comprises a two way protocol such that the computer system is capable of issuing commands to control data collection.

Intermediate device 102 may comprise a data collection system to collect and store data from the wireless transmitter. The data collection system can be configured to communicate periodically with the computer system. The data collection system can transmit data in response to commands from computer system 106 and/or in response to commands from the adherent device.

Activity sensor and activity circuitry 134 can comprise many known activity sensors and circuitry. In many embodiments, the accelerometer comprises at least one of a piezoelectric accelerometer, capacitive accelerometer or electromechanical accelerometer. The accelerometer may comprises a 3-axis accelerometer 134XYZ to generate an accelerometer signal so as to measure at least one of an inclination, a position, an orientation or acceleration of the patient in three dimensions. Work in relation to embodiments of the present invention suggests that three dimensional orientation of the patient and associated positions, for example sitting, standing, lying down, can be very useful when combined with data from other sensors, for example ECG data and/or bioimpedance data, for example a respiration rate of the patient.

Impedance circuitry 136 can generate both hydration data and respiration data. In many embodiments, impedance circuitry 136 is electrically connected to electrodes 112A and 112D and additional electrodes 112B and 112C, as described above, in a four pole configuration, such that electrodes 112A and 112D comprise outer electrodes that are driven with a current and comprise force electrodes that force the current through the tissue. The current delivered between electrodes 112A and 112D generates a measurable voltage between the additional electrodes 112B and 112C, such that the additional electrodes 112B and 112C may comprise inner, sense, electrodes that sense and/or measure the voltage in response to the current from the force electrodes.

ECG circuitry 138 can generate electrocardiogram signals and data from two or more of electrodes 112A and 112D in many ways, for example with an instrumentation amplifier coupled to electrodes 112A and 112D.

FIG. 1D1 shows an electrocardiogram signal 152 that can be measured with ECG circuitry 136. Electrocardiogram signal 152 may comprise several P, Q, R, S and T waves from several heart beats, for example from heart beats from a 1 to 10 minute measurement period. A first heart beat 154 may comprise a first P wave P1, a first Q wave Q1, a first R wave R1, a first S wave S1 and a first T wave T1. A second heart beat 156 may comprise a second P wave P2, a second Q wave Q2, a second R wave R2, a second S wave S2 and a third T wave T2. A heart rate may comprise a number of heart beats per unit time, for example number of hear beats per minute. An interval between heart beats can be used to determine the heart rate. For example the R-R interval corresponding to the period of time between R waves can be used to determine the heart rate. The heart rate variability may comprise a variation in heart rate, for example in response to R-R intervals. Although first heart beat 154 and second heart beat 156 are shown, the ECG signal may comprise several heart beats, for example at least about 10 heart beats, 100 heart beats or even 1000 or more heart beats.

FIG. 1E shows batteries 150 positioned over the flex printed circuit board and electronic components as in FIG. 1D. Batteries 150 may comprise rechargeable batteries that can be removed and/or recharged. In some embodiments, batteries 150 can be removed from the adherent patch and recharged and/or replaced.

FIG. 1F shows a top view of a cover 162 over the batteries, electronic components and flex printed circuit board as in FIGS. 1A to 1E. In many embodiments, an electronics housing 160 may be disposed under cover 162 to protect the electronic components, and in some embodiments electronics housing 160 may comprise an encapsulant over the electronic components and PCB. In some embodiments, cover 162 can be adhered to adherent patch 110 with an adhesive 164 on an underside of cover 162. In many embodiments, electronics housing 160 may comprise a water proof material, for example a sealant adhesive such as epoxy or silicone coated over the electronics components and/or PCB. In some embodiments, electronics housing 160 may comprise metal and/or plastic. Metal or plastic may be potted with a material such as epoxy or silicone.

Cover 162 may comprise many known biocompatible cover, casing and/or housing materials, such as elastomers, for example silicone. The elastomer may be fenestrated to improve breathability. In some embodiments, cover 162 may comprise many known breathable materials, for example polyester, polyamide, and/or elastane (Spandex). The breathable fabric may be coated to make it water resistant, waterproof, and/or to aid in wicking moisture away from the patch.

FIG. 1G shows a side view of adherent device 100 as in FIGS. 1A to 1F. Adherent device 100 comprises a maximum dimension, for example a length 170 from about 2 to 10 inches (from about 50 mm to about 250 mm), for example from about 4 to 6 inches (from about 100 mm to about 150 mm). In some embodiments, length 170 may be no more than about 6 inches (no more than about 150 mm). Adherent device 100 comprises a thickness 172. Thickness 172 may comprise a maximum thickness along a profile of the device. Thickness 172 can be from about 0.2 inches to about 0.4 inches (from about 5 mm to about 10 mm), for example about 0.3 inches (about 7.5 mm).

FIG. 1H shown a bottom isometric view of adherent device 100 as in FIGS. 1A to 1G. Adherent device 100 comprises a width 174, for example a maximum width along a width profile of adherent device 100. Width 174 can be from about 1 to about 4 inches (from about 25 mm to 100 mm), for example about 2 inches (about 50 mm).

FIGS. 1I and 1J show a side cross-sectional view and an exploded view, respectively, of adherent device 100 as in FIGS. 1A to 1H. Device 100 comprises several layers. Gel 114A, or gel layer, is positioned on electrode 112A to provide electrical conductivity between the electrode and the skin. Electrode 112A may comprise an electrode layer. Adhesive patch 110 may comprise a layer of breathable tape 110T, for example a known breathable tape, such as tricot-knit polyester fabric. An adhesive 116A, for example a layer of acrylate pressure sensitive adhesive, can be disposed on underside 110A of adherent patch 110. A gel cover 180, or gel cover layer, for example a polyurethane non-woven tape, can be positioned over patch 110 comprising the breathable tape. A PCB layer, for example flex printed circuit board 120, or flex PCB layer, can be positioned over gel cover 180 with electronic components 130 connected and/or mounted to flex printed circuit board 120, for example mounted on flex PCB so as to comprise an electronics layer disposed on the flex PCB layer. In many embodiments, the adherent device may comprise a segmented inner component, for example the PCB may be segmented to provide at least some flexibility. In many embodiments, the electronics layer may be encapsulated in electronics housing 160 which may comprise a waterproof material, for example silicone or epoxy. In many embodiments, the electrodes are connected to the PCB with a flex connection, for example trace 123A of flex printed circuit board 120, so as to provide strain relief between the electrodes 112A and 112D and the PCB. Gel cover 180 can inhibit flow of gel 114A and liquid. In many embodiments, gel cover 180 can inhibit gel 114A from seeping through breathable tape 110T to maintain gel integrity over time. Gel cover 180 can also keep external moisture, for example liquid water, from penetrating though the gel cover into gel 114A while allowing moisture vapor from the gel, for example moisture vapor from the skin, to transmit through the gel cover. In many embodiments, cover 162 can encase the flex PCB and/or electronics and can be adhered to at least one of the electronics, the flex PCB or adherent patch 110, so as to protect at least the electronics components and the PCB. Cover 162 can attach to adhesive patch 110 with adhesive 1116B. Cover 162 can comprise many known biocompatible cover materials, for example silicone. Cover 162 can comprise an outer polymer cover to provide smooth contour without limiting flexibility. In many embodiments, cover 162 may comprise a breathable fabric. Cover 162 may comprise many known breathable fabrics, for example breathable fabrics as described above. In some embodiments, the breathable cover may comprise a breathable water resistant cover. In some embodiments, the breathable fabric may comprise polyester, nylon, polyamide, and/or elastane (Spandex) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

The breathable cover 162 and adherent patch 110 comprises breathable tape can be configured to couple continuously for at least one week the at least one electrode to the skin so as to measure breathing of the patient. The breathable tape may comprise the stretchable breathable material with the adhesive and the breathable cover may comprises a stretchable material connected to the breathable tape, as described above, such that both the adherent patch and cover can stretch with the skin of the patient. Arrows 182 show stretching of adherent patch 110, and the stretching of adherent patch can be at least two dimensional along the surface of the skin of the patient. As noted above, connectors 122A and 122D between PCB 130 and electrodes 112A and 112D may comprise insulated wires that provide strain relief between the PCB and the electrodes, such that the electrodes can move with the adherent patch as the adherent patch comprising breathable tape stretches. Arrows 184 show stretching of cover 162, and the stretching of the cover can be at least two dimensional along the surface of the skin of the patient. For example, cover 162 and adhesive patch 110 can stretch in two dimensions along length 170 and width 174 with the skin of the patient, and stretching along length 170 can increase spacing between electrodes. Stretching of the cover and adhesive patch 110, for example in two dimensions, can extend the time the patch is adhered to the skin as the patch can move with the skin such that the patch remains adhered to the skin. Cover 162 can be attached to adherent patch 110 with adhesive 116B such that cover 162 stretches and/or retracts when adherent patch 110 stretches and/or retracts with the skin of the patient, for example along two dimensions comprising length 170 and width 174. Electronics housing 160 can be smooth and allow breathable cover 162 to slide over electronics housing 160, such that motion and/or stretching of cover 162 is slidably coupled with housing 160. The printed circuit board can be slidably coupled with adherent patch 110 that comprises breathable tape 110T, such that the breathable tape can stretch with the skin of the patient when the breathable tape is adhered to the skin of the patient. Electronics components 130 can be affixed to printed circuit board 120, for example with solder, and the electronics housing can be affixed over the PCB and electronics components, for example with dip coating, such that electronics components 130, printed circuit board 120 and electronics housing 160 are coupled together. Electronics components 130, printed circuit board 120, and electronics housing 160 are disposed between the stretchable breathable material of adherent patch 110 and the stretchable water resistant material of cover 160 so as to allow the adherent patch 110 and cover 160 to stretch together while electronics components 130, printed circuit board 120, and electronics housing 160 do not stretch substantially, if at all. This decoupling of electronics housing 160, printed circuit board 120 and electronic components 130 can allow the adherent patch 110 comprising breathable tape to move with the skin of the patient, such that the adherent patch can remain adhered to the skin for an extended time of at least one week, for example two or more weeks.

An air gap 169 may extend from adherent patch 110 to the electronics module and/or PCB, so as to provide patient comfort. Air gap 169 allows adherent patch 110 and breathable tape 110T to remain supple and move, for example bend, with the skin of the patient with minimal flexing and/or bending of printed circuit board 120 and electronic components 130, as indicated by arrows 186. Printed circuit board 120 and electronics components 130 that are separated from the breathable tape 110T with air gap 169 can allow the skin to release moisture as water vapor through the breathable tape, gel cover, and breathable cover. This release of moisture from the skin through the air gap can minimize, and even avoid, excess moisture, for example when the patient sweats and/or showers.

The breathable tape of adhesive patch 110 may comprise a first mesh with a first porosity and gel cover 180 may comprise a breathable tape with a second porosity, in which the second porosity is less than the first porosity to minimize, and even inhibit, flow of the gel through the breathable tape. The gel cover may comprise a polyurethane film with the second porosity.

In many embodiments, the adherent device comprises a patch component and at least one electronics module. The patch component may comprise adhesive patch 110 comprising the breathable tape with adhesive coating 116A, at least one electrode, for example electrode 114A and gel 114. The at least one electronics module can be separable from the patch component. In many embodiments, the at least one electronics module comprises the flex printed circuit board 120, electronic components 130, electronics housing 160 and cover 162, such that the flex printed circuit board, electronic components, electronics housing and cover are reusable and/or removable for recharging and data transfer, for example as described above. In many embodiments, adhesive 116B is coated on upper side 110A of adhesive patch 110B, such that the electronics module can be adhered to and/or separated from the adhesive component. In specific embodiments, the electronic module can be adhered to the patch component with a releasable connection, for example with Velcro™, a known hook and loop connection, and/or snap directly to the electrodes. Two electronics modules can be provided, such that one electronics module can be worn by the patient while the other is charged, as described above. For example, about 12 patches can be used to monitor the patient for at least 90 days with at least one electronics module, for example with two reusable electronics modules.

At least one electrode 112A can extend through at least one aperture 180A in the breathable tape 110 and gel cover 180.

In some embodiments, the adhesive patch may comprise a medicated patch that releases a medicament, such as antibiotic, beta-blocker, ACE inhibitor, diuretic, or steroid to reduce skin irritation. The adhesive patch may comprise a thin, flexible, breathable patch with a polymer grid for stiffening. This grid may be anisotropic, may use electronic components to act as a stiffener, may use electronics-enhanced adhesive elution, and may use an alternating elution of adhesive and steroid.

FIG. 1K shows at least one electrode 190 configured to electrically couple to a skin of the patient through a breathable tape 192. In many embodiments, at least one electrode 190 and breathable tape 192 comprise electrodes and materials similar to those described above. Electrode 190 and breathable tape 192 can be incorporated into adherent devices as described above, so as to provide electrical coupling between the skin and electrode through the breathable tape, for example with the gel.

Second adherent device 100J and third adherent device 100A may comprise components similar to adherent device 100, described above. The processor of adherent device 100, described above may comprise a system controller to control communication and/or actions of first adherent device 100J and second device 100A, for example data collection and transmission. In many embodiments, data collected from second adherent device 100J and third adherent device 100A is sent wirelessly to device 100, which device 100 transmits the data to the intermediate device.

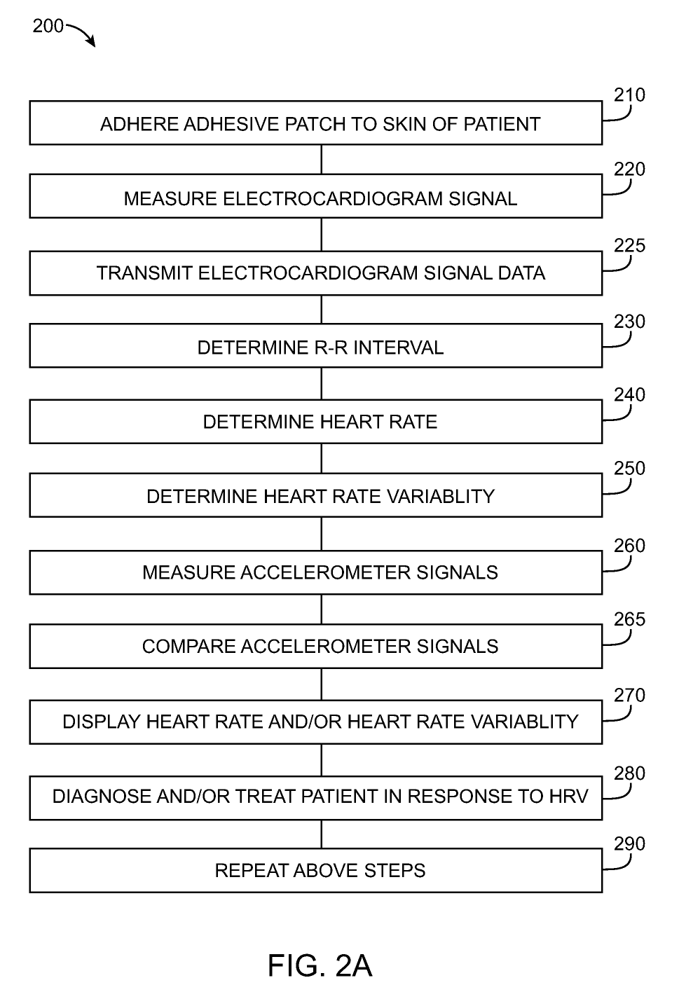

FIG. 2A shows a method 200 of determining heart rate variability of a patient. Method 200 can be performed with adherent patch and the processor system, as described above.

A step 210 adheres an adhesive patch to the skin of the patient, for example a patch as described above. The patch can be adhered to the patient for an extended period comprising at least one week, such that measurements can be taken from electrodes of the patch for the extended period of at least one week.

A step 220 measures the electrocardiogram signal when the patch is adhered to the patient. The electrocardiogram signal can be measured for a period from about one to ten minutes. The electrocardiogram signal can be stored with the processor on the adherent patch, as described above.

A step 225 transmits the data from the adherent device to the computer system. The data may comprise at least one of the ECG signal or information derived from the ECG signal, such as R-R intervals and/or frequency information. The data can be transmitted from the adherent device to the intermediate device. In some embodiments, for example in a chiropractor's office, the data can be transmitted from the adherent device to the computer system with a wireless signal, for example 802.11 compliant wireless transmission from the adherent device to wireless circuitry on the computer system.

A step 230 determines an R-R interval. Each heat beat may comprise P, Q, R, S and T waves that correspond to known physiology of the electrocardiogram signal. The R-R interval corresponds to the rate of the heart beat and can be determined in many ways. The processor can store the ECG signal, for example with analog to digital conversion, and transfer the signal data to the intermediate device. At least one of the intermediate device or the processor system can calculate the R-R interval for several heart beats measured for the period of about one to ten minutes from the digital signal data. In some embodiments, the processor on the adherent patch can determine the R-R interval and store the R-R interval for transmission to the computer system.

A step 240 determines the heart rate. The heart rate can be determined from the R-R interval for several heart beats, as described above. The heart rate may comprise an average heart rate from several R-R intervals from several heart beats. The heart rate can be determined with calculations from at least one of the processor on the patch, the intermediate device or the computer system.

A step 250 determines the heart rate variability. The heart variability can be determined in many ways, for example with at least one of a time domain determination, a frequency domain determination or a non-linear determination. The heart rate variability can be determined with time domain calculations. The time domain determination may comprise calculations based on a standard deviation of R-R heart rate intervals, for example a standard deviation of average R-R intervals. The heart rate variability can be determined with frequency domain calculations, for example with a ratio of ratio of low-to-high frequency spectra power (LF/HF) as described above. The heart rate variability can be determined with non-linear calculations, for example with Poincaré analysis as described above.

A step 260 measures at least one accelerometer signal. The at least one accelerometer signal may comprise a signal from an accelerometer mounted to measure rotation and/or flexion extension of the patient, for example an accelerometer attached to the head of the patient. The at least one accelerometer signal may comprise two accelerometer signals, for example a first accelerometer connected to the thorax of the patient and a second accelerometer connected to the head of the patient to measure relative rotation of the first accelerometer to the second accelerometer. The at least one accelerometer signal, may comprise at least three, or more, accelerometer signals to determine at least one of rotation, flexion/extension or lateral movement of at least one of a back or neck of the patient.

A step 265 compares the accelerometer signals. Accelerometer signals can be compared to determine at least one of a rotation, a flexion extension or lateral movement of at least one of a back or neck of the patient. The accelerometers can be positioned on the patient as described above, and signals can be measured to determine the patient range of motion. For example, a first accelerometer signal can be measured with the head in a first position and a second accelerometer signal can be measured with the head in a second position to determine a range of movement of rotation of the head of the patient. For example, a rotational range of motion of the head can be measured with rotation of the head between the first position and the second position. A similar range of motion can be determined for each of flexion/extension and lateral movement. In some embodiments, a first accelerometer signal from an accelerometer at a first location, for example on the head of the patient, can be compared to a second accelerometer at a second location, for example on the thorax of the patient, to determine the range of motion between the two accelerometers. For example, a first accelerometer could be positioned on the lower back of the patient and the second accelerometer positioned on the upper back of the patient, to determine the range of motion of the back between the first accelerometer and the second accelerometer.

The accelerometer signals can be compared to determine how long a patient sits and/or sitting posture of the patient.

A step 270 displays the heart rate and/or heart rate variability. The heart rate and/or heart rate variability can be displayed in many ways to the treating health care provider, for example on the display caregiver computer system, with a printout on paper, with a display on a hand held device.

A step 280 diagnoses and/or treats the patient in response to at least one of the heart rate variability or the accelerometer signal. The patient can be treated in many ways, for example with chiropractic adjustment.

A step 290 may repeat at least some of the above steps. The ECG signal can be measured at least a second time over at least one week when the patch is continuously adhered to the skin of the patient. At least a second patch can be adhered to the skin, for example after one week to adhere to the skin of the patient. The heart rate variability can be determined many

The processor system, as described above, can be configured to perform the method 200, including many of the steps described above. It should be appreciated that the specific steps illustrated in FIG. 2A provide a particular method of monitoring heart rate variability of a patient, according to one embodiment of the present invention. Other sequences of steps may also be performed according to alternative embodiments. For example, alternative embodiments of the present invention may perform the steps outlined above in a different order. Moreover, the individual steps illustrated in FIG. 2A may include multiple sub-steps that may be performed in various sequences as appropriate to the individual step. Furthermore, additional steps may be added or removed depending on the particular applications. One of ordinary skill in the art would recognize many variations, modifications, and alternatives.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An adherent device for chiropractic monitoring of a patient, the device comprising:
an adhesive patch to adhere to a skin of the patient;
at least two electrodes connected to the patch and capable of electrically coupling to the patient;
electrocardiogram circuitry coupled to the at least two electrodes to measure an electrocardiogram signal of the patient; and
a processor comprising a tangible medium coupled to the electrocardiogram circuitry, the processor comprising a tangible medium configured to determine at least one of a heart rate or a heart rate variability of the patient in response to the electrocardiogram signal.

2. The adherent device of claim 1 wherein the adhesive patch is configured to mechanically couple the at least two electrodes to the skin and obtain the electrocardiogram signal for at least one week.

3. The adherent device of claim 2 wherein the processor is configured to determine the heart rate variability with at least one of a time domain determination, a frequency domain determination or a non-linear determination.

4. The adherent device of claim 3 wherein the processor is configured to determine the heart rate variability with the frequency domain determination in response to at least one of a low frequency from about 0.04 to 0.15 Hz or a high frequency from about 0.15 Hz to about 0.4 Hz.

5. The adherent device of claim 3 wherein the processor is configured to determine the heart rate variability with the frequency domain determination in response to a ratio of a low frequency band comprising at least one low frequency from about 0.04 to 0.15 Hz and a high frequency band comprising at least one high frequency from about 0.15 Hz to about 0.4 Hz.

6. The adherent device of claim 3 wherein the processor is configured to determine the heart rate variability with the time domain determination in response to a standard deviation of R-R intervals.

7. The adherent device of claim 2 wherein the processor is configured to determine R-R intervals based on from about one to ten minutes of the electrocardiogram signal and wherein the heart rate variability comprises a standard deviation of the R-R intervals and wherein the processor is configured to determine the heart rate variability several times over the at least one week.

8. The adherent device of claim 2 wherein the processor is configured to determine averages of R-R intervals from the electrocardiogram signal and wherein the processor is configured to determine each of the averages of the R-R intervals based on from about one to ten minutes of the electrocardiogram signal and wherein the heart rate variability comprises a standard deviation of the averages of the R-R intervals and wherein the processor is configured to determine the heart rate variability several times over the at least one week.

9. The adherent device of claim 2 wherein the processor is configured to determine the heart rate variability at least once per hour for each hour of the at least one week.

10. The adherent device of claim 1 wherein the adhesive patch is mechanically coupled to the at least two electrodes and the electrocardiogram circuitry to support the at least two electrodes and the electrocardiogram circuitry when the adherent patch is adhered to the skin of the patient.

11. The adherent device of claim 1 further comprising wireless communication circuitry to transmit the heart rate variability to a caregiver computer system with a communication protocol.

12. The adherent device of claim 11 wherein the communications protocol comprises a two way protocol such that the caregiver computer system is capable of issuing commands to the processor to control data collection.

13. The adherent device of claim 12 wherein the processor is configured to transmit the at least one of the heart rate or the heart rate variability to the caregiver computer system in response to a command from the caregiver computer system when the wireless communication circuitry is located in an office of the caregiver.

14. The adherent device of claim 11 wherein the caregiver computer system comprises a display visible to a caregiver and a tangible medium configured to show information on the display in response to the electrocardiogram signal.

15. The adherent device of claim 11 wherein the wireless communication circuitry is configured to communicate with a remote center using an intermediate device.

16. The adherent device of claim 1 further comprising wireless communication circuitry to transmit the at least one of the heart rate or the heart rate variability to a remote center with a communication protocol.

17. The adherent device of claim 16 wherein the wireless communication circuitry is configured to transmit the electrocardiogram signal to the remote center with an intermediate device.

18. The adherent device of claim 17 wherein the communication protocol comprises at least one of Bluetooth, Zigbee, WiFi, WiMax, IR, a cellular protocol, amplitude modulation or frequency modulation.

19. The adherent device of claim 17 wherein the intermediate device comprises a data collection system to collect and/or store data from the wireless transmitter and wherein the data collection system is configured to communicate periodically with the remote center with wireless connection and/or wired communication.

20. The adherent device of claim 17 wherein the communications protocol comprises a two way protocol such that the remote center is capable of issuing commands to the processor to control data collection.

21. The adherent device of claim 17 wherein the processor is configured to control collection and transmission of data from the electrocardiogram circuitry.

22. The adherent device of claim 1 wherein the adherent patch comprises a breathable tape, the breathable tape comprising a breathable material with an adhesive.

23. The adherent device of claim 1 further comprising an accelerometer connected to the adhesive patch to measure at least one of a rotation, a flexion/extension, a lateral movement or a posture of the patient.

24. The adherent device of claim 1 further comprising an accelerometer connected to a second adhesive patch configured for placement on at least one of a head, a neck or an ear of the patient.

25. An adherent device system for chiropractic monitoring of a patient, the system comprising:
at least one adhesive patch to adhere to a skin of the patient;
at least accelerometer connected to the at least one patch to generate at least one accelerometer signal; and
a processor coupled to the at least one accelerometer, the processor comprising a tangible medium configured to determine at least one of a rotation, a flexion/extension, a lateral movement or a posture of the patient in response to the at least one accelerometer signal.

26. A method of monitoring a patient, the method comprising:
adhering an adhesive patch to a skin of the patient to couple at least two electrodes to the skin of the patient;
measuring an electrocardiogram signal of the patient with electrocardiogram circuitry coupled to at least two of the at least two electrodes; and
determining at least one of a heart rate or a heart rate variability of the patient.

27. The method of claim 26 wherein the patch is adhered to the patient for at least one week and the heart rate or the heart rate variability is determined for the at least one week.

28. The method of claim 27 wherein averages of R-R intervals are determined from the electrocardiogram signal and each of the averages of the R-R intervals are determined based on from about one to ten minutes of the electrocardiogram signal and wherein the heart rate variability comprises a standard deviation of the averages of the R-R intervals and wherein the heart rate variability is determined several times over the at least one week.

29. The method of claim 27 wherein the heart rate variability is determined at least once per hour for each hour of the at least one week.

30. The method of claim 26 wherein the adhesive patch supports the at least two electrodes and the processor when the adherent patch is adhered to the skin of the patient.

31. A method of chiropractic monitoring a patient, the method comprising:
adhering at least one adhesive patch to a skin of the patient to couple at least one accelerometer to the skin of the patient;
measuring at least one accelerometer signal of the patient with the at least one accelerometer coupled to the skin of the patient; and
determining at least one of a rotation, a flexion/extension, a lateral movement or a posture of the patient in response to the accelerometer signal.

32. An adherent device to monitor a patient for an extended period, the device comprising:
a breathable tape comprising a porous material with an adhesive coating to adhere the breathable tape to a skin of the patient;
at least one electrode affixed to the breathable tape and capable of electrically coupling to a skin of the patient;
at least one gel disposed over a contact surface of the at least one electrode to electrically connect the electrode to the skin;
a circuit board connected to the electrodes to couple the printed circuit board to the electrodes;
electronic components electrically connected to the printed circuit board and coupled to the at least one electrode to measure an electrocardiogram signal of the patient; and
a processor coupled to the electronic components to determine at least one of a heart rate or a heart rate variability of the patient.

33. The adherent device of claim 32 further comprising a breathable cover disposed over the circuit board and electronic components and connected to at least one of the electronics components, the printed circuit board or the breathable tape.

34. The adherent device of claim 33 further comprising an electronics housing adhered to at least one of the electronics components or the printed circuit board, such that the electronics housing is disposed between the cover and electronics components.

35. The adherent device of claim 32 further comprising a gel cover positioned over the breathable tape to control hydration of the at least one gel and to inhibit a flow of the gel through the breathable tape and wherein the printed circuit board is located over the gel cover such that the gel cover is disposed between the breathable tape and the printed circuit board.

36. The adherent device of claim 32 further comprising a gel cover and wherein the breathable tape comprises a first porosity and the gel cover comprises a breathable tape with a second porosity, the second porosity less than the first porosity to decrease a flow of moisture to and from the at least one gel and to decrease flow of the gel through the breathable tape.

37. The adherent device of claim 32 wherein the breathable tape, the adhesive coating and the at least one electrode are separable from the printed circuit board and electronic components such that the printed circuit board, electronic components, housing and cover are reusable.

38. The adherent device of claim 32 wherein the at least one electrode extends through at least one aperture in the breathable tape.
